(19) United States
(12) Patent Application Publication
(10) Pub. No.: US 2026/0248386 A1
(43) Pub. Date: Aug. 27, 2026

(54) HANDHELD OPTICAL MEASUREMENT APPARATUS AND METHOD OF ALIGNING A HANDHELD OPTICAL MEASUREMENT APPARATUS

(71) Applicant: Occuity Limited, London (GB)

(72) Inventors: Robin TAYLOR, Berkshire (GB); James REYNOLDS, Gloucestershire (GB); Piotr LASZCZAK, Warszawa (PL); Piotr WALASZEK, Szczaniec (PL)

(73) Assignee: Occuity Limited, London (GB)

(21) Appl. No.: 18/874,690

(22) PCT Filed: Jun. 6, 2023
(86) PCT No.: PCT/EP2023/065158

§ 371 Date: Dec. 13, 2024

(30) Foreign Application Priority Data
Jun. 14, 2022 (GB) GB2208731.6

(51) Int. Cl. A61B 3/15; A61B 3/00; A61B 3/10

(52) U.S. Cl. CPC A61B 3/152; A61B 3/0008; A61B 3/0041; A61B 3/0091; A61B 3/1005

(57) ABSTRACT

A handheld optical measurement apparatus (100) comprises an optical measurement system (200, 201) comprising an optical reception path (202), a display device (116), and a housing (101) comprising a longitudinal measurement axis (113) and the optical measurement system (200, 201), the optical reception path (202) being coaxial with the longitudinal measurement axis (113). An alignment system is also provided comprising an extended light source (110) configured as a predetermined shape and mounted on the housing (101) and disposed off-axis with respect to the longitudinal measurement axis (113), the extended light source being configured to illuminate a reflective target. An optical sensor device (112) is mounted on the housing (101) off-axis relative to the longitudinal measurement axis (113) and configured to receive light reflected by the reflective target and to capture an image comprising the light. A processing resource (208, 210, 212, 214, 216) is operably coupled to the optical sensor device (112) and the display device (116). The processing resource is configured to display on the display device (116) the image captured by the optical sensor device (112) and substantially contemporaneously display an alignment reference (220) on the display device (116), the alignment reference (220) being fixed in position and size, and providing, when in use, a reference to facilitate manual alignment of the optical measurement system (200, 201) with the reflective target. The processing resource is also configured to assess alignment of the optical measurement system (200, 201) with respect to fidelity of shape and centrality of the reflected light in the image captured by the optical sensor device (112).

The present invention relates to a handheld optical measurement apparatus of the type that, for example, is held to reflective target, such as an eye, for measurement of a property of the reflective target. The present invention also relates to a method of aligning a handheld optical measurement apparatus, the method being of the type that, for example, aligns the apparatus with a reflective target, such as an eye, for measurement of a property of the reflective target.

In the field of metrology, it is known to provide handheld optical measurement apparatuses. However, a significant challenge when designing a handheld optical measurement apparatus is alignment of the apparatus with respect to the reflective target, for example the eye, when the apparatus is offered to the eye for performance of a measurement. One such optical measurement apparatus is a pachymeter, which is used to measure the thickness of a cornea of an eye. In order to measure corneal thickness successfully, a confocal optical axis of the apparatus should be aligned with respect to the eye in, for example, 3 Cartesian axes, and rotation about two of the axes (pitch and yaw).

When light is scanned through a cornea, the amount of corneal tissue through which electromagnetic radiation has to be focussed to pass through the cornea varies depending upon the position of the confocal optical axis along which the electromagnetic radiation is focussed. In this regard, off-centre measurement with respect to the X or Y directions can result in increased distances being observed between the anterior and posterior interfaces of the cornea along the confocal optical axis. Indeed, the centres of curvature of the anterior and posterior interfaces of the cornea are not typically coincident, leading to greater thicknesses being observed at off-centre locations of the cornea. Furthermore, where electromagnetic radiation, scanned through the cornea along the confocal optical axis, is incident upon an interface of the cornea that is not normal to the confocal optical axis, received reflections of the incident electromagnetic radiation are attenuated and hence strength of received signal is reduced.

Therefore, for the sake of consistency and accuracy, measurement is performed where the confocal optical axis passes through the centre of the cornea.

Additionally, when measuring thickness confocally, a confocal measurement arrangement can typically only focus a beam of electromagnetic radiation within a finite range of locations along the confocal optical axis, the finite range of locations constituting scanning range. Therefore, to measure corneal thickness, for example, both the anterior and posterior interfaces of the cornea have to be within the scanning (Z) range of the confocal measurement unit.

Furthermore, the alignment of the confocal measurement apparatus in yaw and pitch is desirable to minimise so-called “cosine errors” when measuring thickness and thereby ensuring the central corneal thickness is targeted accurately for measurement.

According to a first aspect of the present invention, there is provided a handheld optical measurement apparatus, the apparatus comprising: an optical measurement system comprising an optical reception path; a display device; a housing comprising a longitudinal measurement axis and the optical measurement system, the optical reception path being coaxial, at least in part, with the longitudinal measurement axis; and an alignment system comprising: an extended light source configured as a predetermined shape and mounted on the housing and disposed off-axis with respect to the longitudinal measurement axis, the extended light source being configured to illuminate, when in use, a reflective target; an optical sensor device mounted on the housing off-axis relative to the longitudinal measurement axis and configured to receive, when in use, light reflected by the reflective target and to capture an image comprising the light; and a processing resource operably coupled to the optical sensor device and the display device; wherein the processing resource is configured to display on the display device the image captured by the optical sensor device and substantially contemporaneously display an alignment reference on the display device, the alignment reference being fixed in position, and providing, when in use, a reference to facilitate manual alignment of the optical measurement system with the reflective target; and the processing resource is configured to assess alignment of the optical measurement system with respect to fidelity of shape and centrality of the reflected light in the image captured by the optical sensor device.

The size of the alignment reference may be fixed.

The optical measurement system may be configured to make a measurement in response to the assessment of alignment.

The processing resource may be arranged to analyse the image comprising the reflected light captured by the optical sensor device and to identify a plurality of boundary pixels of the reflected light.

The plurality of boundary pixels may be a plurality of inner boundary pixels of the reflected light. The plurality of boundary pixels may be pixels of increased illuminance as compared with respective illuminances of another plurality of respective neighbouring pixels.

The processing resource may be configured to define a central reference line within the image comprising the reflected structured light captured and to measure a plurality of perpendicular distances from the central reference line to the plurality of boundary pixels, respectively.

The central reference line may be a vertical reference line.

The processing resource may be arranged to model a boundary line defined by the plurality of boundary pixels and assess the centrality, size and fidelity of shape of the boundary line.

The processing resource may be arranged also to assess the size of shape of the boundary line.

The processing resource may be configured to use the plurality of perpendicular distances to fit a shape to the boundary line.

The processing resource may be configured to determine whether respective luminous intensities of the plurality of boundary pixels satisfy a predetermined threshold criterion.

The processing resource may be configured to analyse a plurality of sets of substantially parallel pixel positions; the plurality of sets of parallel pixel positions may have respective predetermined spacings therebetween.

The plurality of sets of substantially parallel pixel positions may be offset with respect to each other.

Each set of the plurality of sets of substantially parallel pixel positions may be parallel with respect to each other. Each set of the plurality of sets of substantially parallel pixel positions may extend vertically.

The plurality of sets of substantially parallel pixel positions may be arranged to correspond to expected locations of a first peripheral side of the reflected light, a second peripheral side of the reflected structured light and a position between the first and second peripheral sides; the second peripheral side may be opposite the first peripheral side.

The expected locations of the first peripheral side, the second peripheral side and the position in between the first and second peripheral sides may correspond to an aligned state of the optical measurement system with the reflective target.

The plurality of sets of substantially parallel pixel positions may comprise: an outer boundary set of pixel positions; an inner boundary set of pixel positions; and an intermediate set of pixel positions between the first and second boundary sets of pixel positions.

Each of the plurality of sets of substantially parallel pixel positions may comprise a first subset of pixel positions and a second subset of pixel positions; the first and second subsets of pixel positions may be arranged in parallel with respect to each other. The first subset of pixel positions may be arranged linearly. The second subset of pixel positions may be arranged linearly.

The processing resource may be configured to analyse illuminance of pixel positions of each set of the plurality of sets of substantially parallel pixel positions in order to determine whether the pixels of the each set of the plurality of sets of substantially parallel pixel positions satisfy a respective predetermined illuminance threshold criterion.

The predetermined illuminance threshold criterion may be set or adjusted by reference to illuminance of a number of pixels of the image, for example an average of the illuminance of the number of pixels.

The optical measurement system may be configured to make a plurality of distance measurements.

The optical measurement system may be a confocal measurement system. The optical measurement system may be an interferometric measurement system, for example a low-coherence interferometric measurement system. The optical measurement system may be configured to measure distance.

The plurality of measurements may be a plurality of distance measurements to the reflective target.

The apparatus may further comprise: the optical sensor device configured to capture a plurality of images comprising the reflected light; the processing resource may be configured to make a plurality of respective assessments of alignment of the optical measurement system with respect to fidelity of shape and centrality of the reflected light of the plurality of images captured; the plurality of assessments of alignment may respectively correspond to the plurality of distance measurements; wherein the processing resource may be configured to select a measurement of the plurality of measurements in response to an alignment assessment of the plurality of alignment assessments; the alignment assessment may correspond to the measurement of the plurality of measurements.

The alignment assessment of the plurality of alignment assessments may correspond to an aligned state of the optical measurement system with respect to the reflective target.

The apparatus may further comprise: the processing resource configured to indicate the plurality of distance measurements.

The processing resource may be configured to cooperate with the display device to output the plurality of distance measurements.

In accordance with a second aspect of the present invention, there is provided a method of aligning a handheld optical measurement apparatus with a reflective target, the method comprising: an extended light source of a predetermined shape illuminating the reflective target from an off-axis position with respect to a longitudinal measurement axis of a housing of the handheld optical measurement apparatus; receiving light reflected from the reflective target; capturing an image comprising the reflected light received using an optical sensor device mounted on a housing of the handheld optical measurement apparatus and off-axis relative to the longitudinal measurement axis of the housing; displaying the image captured by the optical sensor device and substantially contemporaneously displaying an alignment reference with the image, the alignment reference being fixed in position, and providing, when in use, a reference to facilitate manual alignment of an optical measurement system of the handheld optical measurement apparatus with the reflective target; and assessing alignment of the optical measurement system with respect to fidelity of shape and centrality of the reflected light in the image captured by the optical sensor device.

It is thus possible to provide an apparatus and method capable of enabling an operator to align coarsely a handheld optical measurement apparatus relative to an eye, thereby enabling accurate measurements to be made of a property of the eye, for example corneal thickness. The coarse alignment to the eye enables relatively simple optical vision-based techniques to be employed to align in X and Y axes. Such vision-based techniques attract a low processing overhead and avoid the use of complex and sometimes bulky alignment hardware.

At least one embodiment of the invention will now be described, by way of example only, with reference to the accompanying drawings, in which:

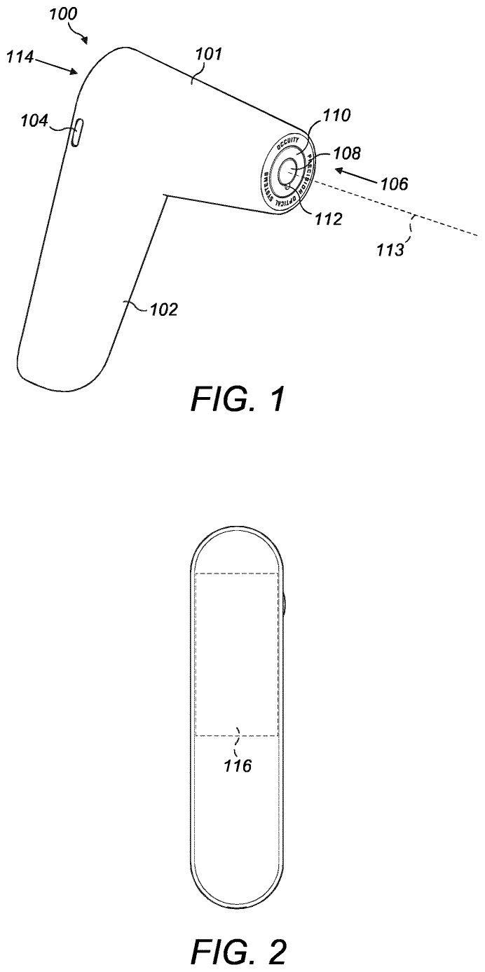

FIG. 1 is perspective schematic diagram of a handheld optical measurement apparatus constituting an embodiment of the invention;

FIG. 2 is a rear elevation of the handheld optical measurement apparatus of FIG. 1;

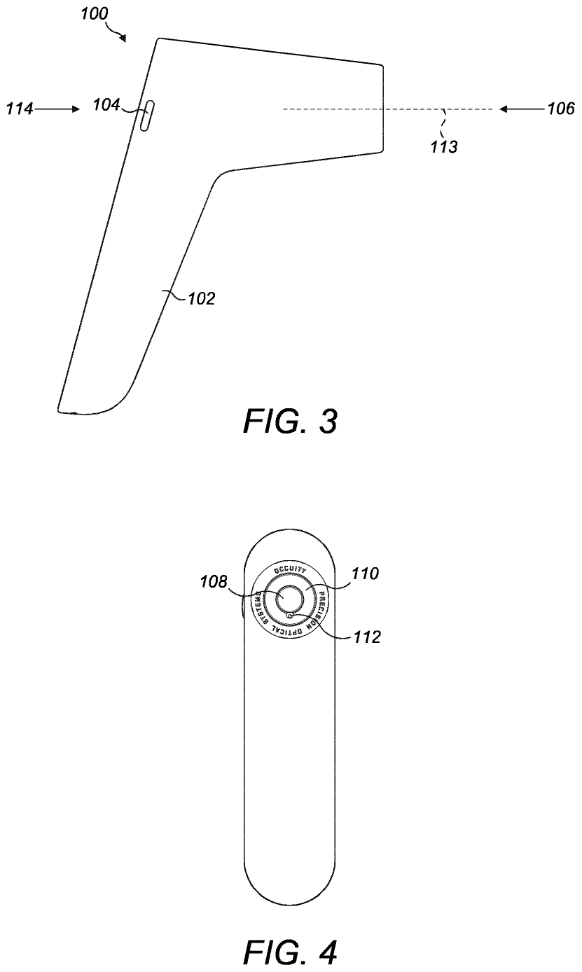

FIG. 3 is a side elevation of the handheld optical measurement apparatus of FIG. 1;

FIG. 4 is a front elevation of the handheld optical measurement apparatus of FIG. 1;

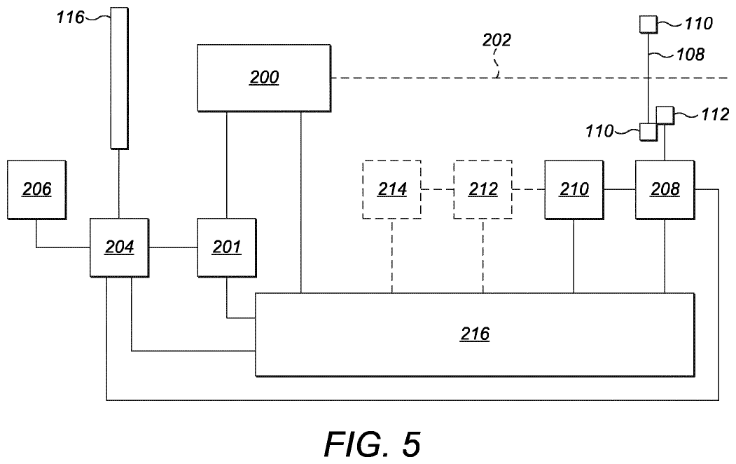

FIG. 5 is a schematic diagram of an alignment and measurement system of the handheld optical measurement apparatus of FIG. 1 constituting another embodiment of the invention;

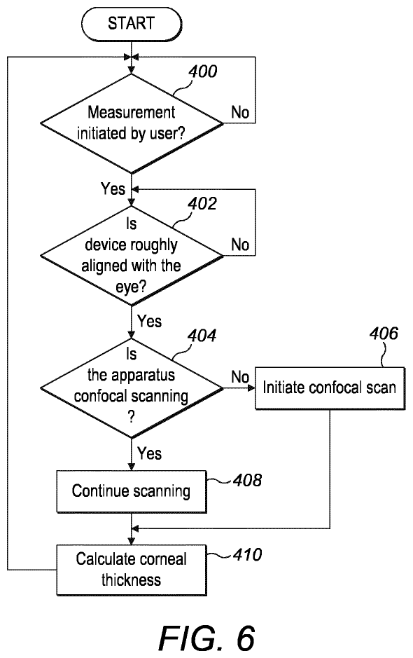

FIG. 6 is a flow diagram of a method of measuring a property of an eye constituting a further embodiment of the invention;

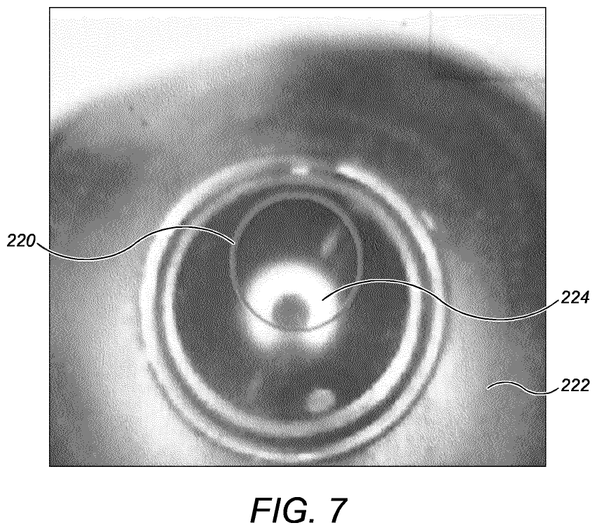

FIG. 7 is a schematic diagram of an image of an eye captured by the apparatus of FIG. 1;

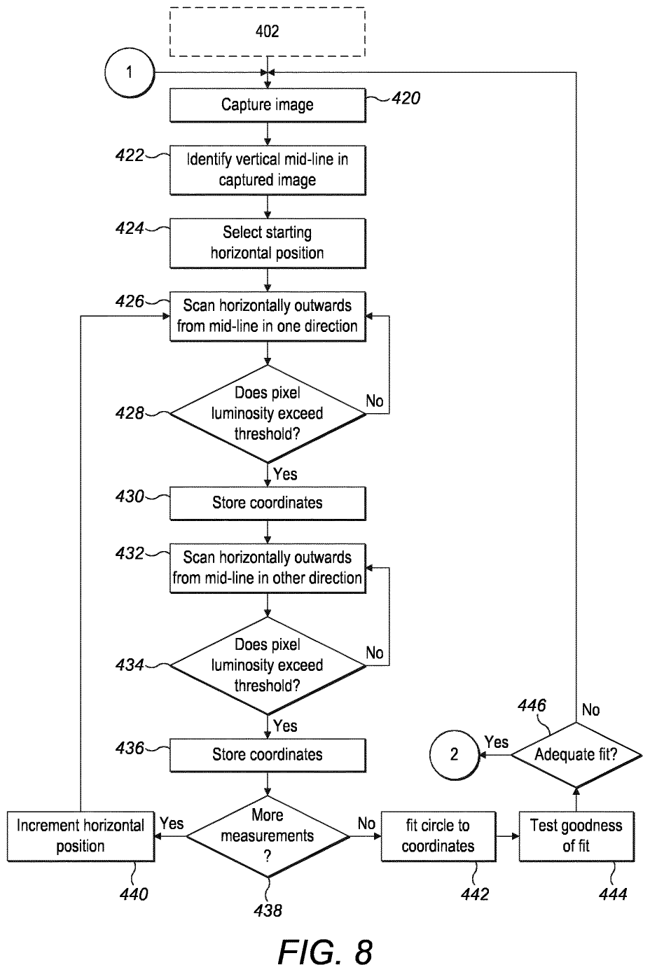

FIG. 8 is a first part of a flow diagram of an automated method of determining X and Y alignment of the apparatus of FIG. 1 and constituting an embodiment of the invention;

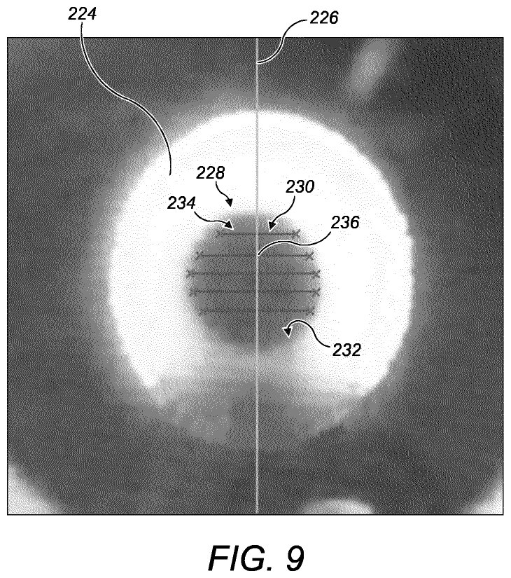

FIG. 9 is an image of an eye augmented with markers to assist visualisation of operation of the method of FIG. 8;

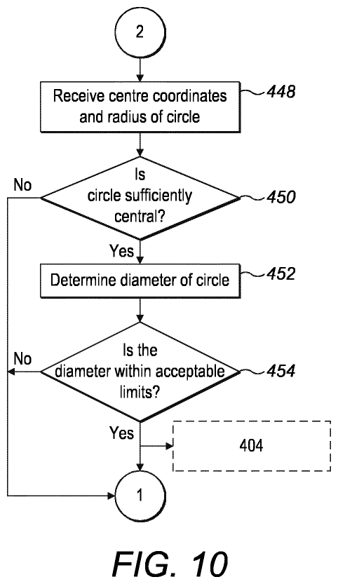

FIG. 10 is a second part of the flow diagram of FIG. 8;

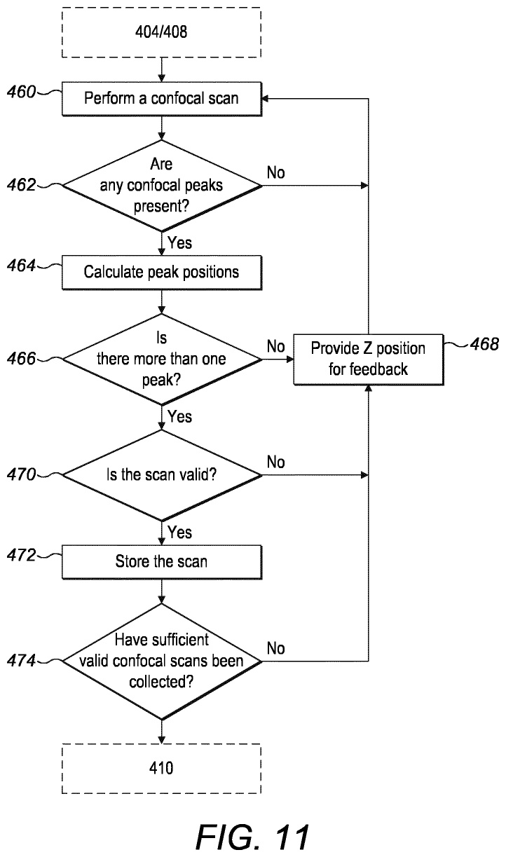

FIG. 11 is a flow diagram of a method of confocal measurement employed by the apparatus of FIG. 1;

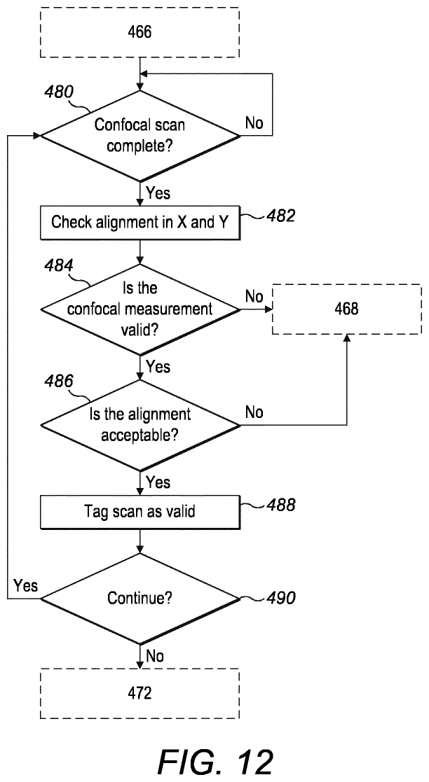

FIG. 12 is a flow diagram of a verification step of FIG. 11 in greater detail;

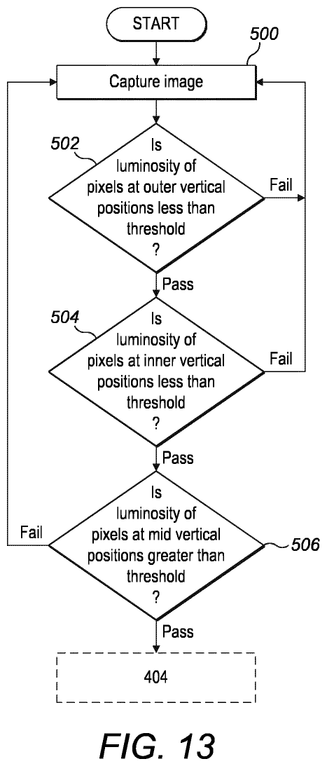

FIG. 13 a flow diagram of another method of determining X and Y alignment of the apparatus of FIG. 1 and constituting another embodiment of the invention;

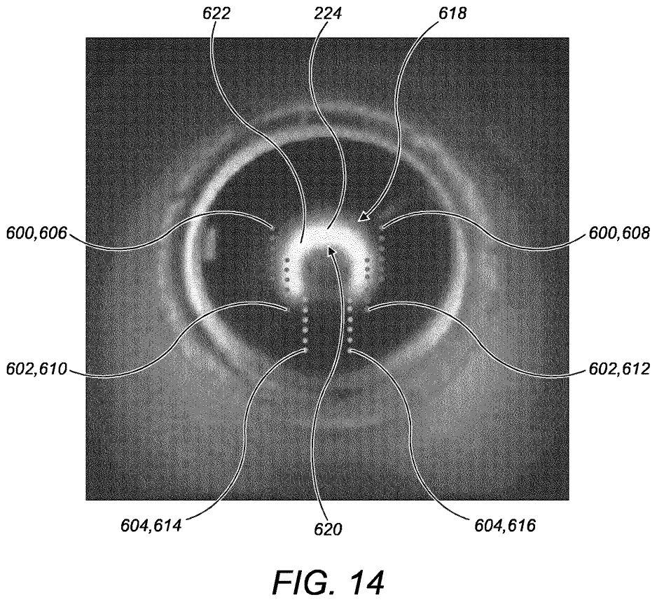

FIG. 14 is an image of an eye augmented with markers to assist visualisation of operation of the method of FIG. 13;

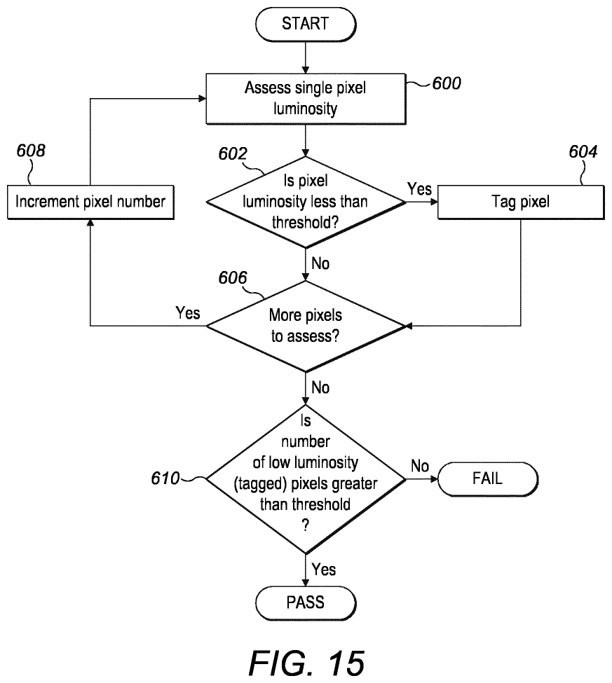

FIG. 15 is a flow diagram of two steps of the method of FIG. 13 in greater detail; and

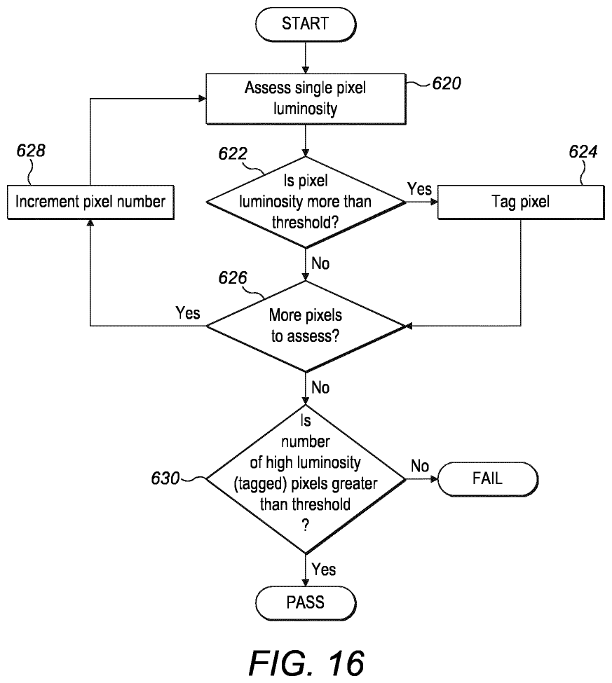

FIG. 16 is a flow diagram of another step of the method of FIG. 13 in greater detail.

Throughout the following description identical reference numerals will be used to identify like parts.

Referring to FIGS. 1 to 4, a handheld optical measurement apparatus 100, for example a handheld pachymeter, comprises a housing 101 having, in this example, a handle portion 102 so that the housing can be gripped and held by an operator. A power button 104 is provided to a side of the housing 101. Although not completely shown in FIGS. 1 to 4, the apparatus 100 comprises an optical measurement system and an alignment system. On a patient-facing side 106 of the pachymeter 100, an optical port 108 is provided. A light source 110 of the alignment system is also provided at the patient-facing side 106 of the pachymeter 100, the light source 110 extending about the optical port 108, the optical port 108 being a circular aperture closed by an optically transmissive window, although the skilled person will appreciate that other shaped ports can be provided. In this example the light source 110 is an array of Light Emitting Diodes (LEDs) configured to emit light in the visible range of the electromagnetic spectrum the light source 110 constituting an extended light source capable of flood illuminating a target, for example a reflective target, such as an eye of a patient. However, the skilled person should appreciate that the light source 110 can be configured in a number of ways, including a continuous light source. The light source 100, in this example, extends completely around the optical port 108. However, in other examples, the light source 100 can extend around a portion of the optical port 108, for example so as to form a substantially horseshoe-like shape. In this regard, any suitable predetermined shape of light source can be employed. The light source 110 is mounted on the housing 101 such that the light source 110 is disposed off-axis with respect to a longitudinal measurement axis 113 of the housing 101. It is desirable that the light source 110 does not intersect the longitudinal measurement axis 113. An optical sensor device of the alignment system, for example a camera 112, is disposed, in this example, adjacent the periphery of the optical port 108, between the light source 110 and the optical port 108. As such, the camera 112 is also mounted on the housing 101 off-centre relative to the longitudinal measurement axis 113.

An operator side 114 of the housing 100 comprises a display device 116 (FIG. 2), for example an LED display.

Referring to FIG. 5, a confocal measurement unit 200 of an optical measurement system is disposed within the housing 101 and configured to emit electromagnetic radiation along a confocal optical axis 202 and receive backscattered electromagnetic radiation along the confocal optical axis 202. The confocal optical axis 202 comprises an optical reception path therealong, which is coaxial, at least in part, with the longitudinal measurement axis 113 mentioned above. The confocal measurement unit 200 is operably coupled to a confocal measurement processor unit 201 of the optical measurement system, the confocal measurement processor unit 201 being operably coupled to a display driver unit 204 of the alignment system, the display driver unit 204 constituting a processing resource. The display driver unit 204 is operably coupled to the display device 116, a reference maker generator unit 206 and an image capture unit 208. The image capture unit 208 is operably coupled to a boundary analysis unit 210. In this example, a curve fitting engine 212 is operably coupled to the boundary analysis unit 210 and an XY determination unit 214. The curve fitting engine 212 and the XY determination unit 214 are provided specifically for the method being employed in one embodiment in order to determine X and Y alignment of the optical axis 202 of the confocal measurement unit 200 with an eye of a patient. In this example, the processing resource therefore comprises the boundary analysis unit 210, the curve fitting engine 212, the XY determination unit 214 and a controller 216. However, in other embodiments, the curve fitting engine 212 and the XY determination unit 214 need not be employed. The controller 216 is operably coupled to the confocal measurement unit 200, the confocal measurement processor unit 201, the display driver unit 204, the image capture unit 208, the boundary analysis unit 210, the curve fitting engine 212, and the XY determination unit 214.

In this example, and some others, the camera 112 is arranged so that an optical axis thereof relative to a frame of reference thereof intersects with the confocal optical axis 202 at or close to, for example less than 20 mm from, an approximate working distance of about 19 mm for the measurement apparatus 100, for example a centre of a scan range of the confocal measurement unit 200. Such intersection can be achieved mechanically by design or by defining a centre of the frame of reference in software and performing all calculations of X and Y relative to that point.

In operation (FIGS. 6 to 16), the pachymeter 100 is powered up by pressing the power button 104. Once the hardware and software of the pachymeter 100 has initialised, the pachymeter 100 awaits (Step 400) initiation of measurement by an operator, which can be the patient in some examples, or in this example a separate operator, for example a physician. In this regard, the display device 116 is a touch-sensitive display device and the display driver 204 presents a “Start measurement” button on the display device 116 for the operator to press to indicate that the pachymeter 100 has been brought sufficiently close to the patient's eye so that the camera 112 can capture an image of the patient's eye and that the operator is ready for measurement to begin. Once the “Start measurement button” is pressed, the light source 110 and the camera 112 are activated. To determine whether the pachymeter 100 is sufficiently close to the patient's eye and coarsely aligned in X and Y axes, the display device 116 is provided with real-time images of the patient's eye captured by the camera 112 in cooperation with the image capture unit 208 so that the operator can determine when the patient's eye is within the field of view of the camera 112. The operator therefore offers the patient-facing side 106 of the pachymeter 100 to the eye of the patient until an image of the eye, captured in real time by the camera 112, is seen on the display 116. Additionally, the reference marker unit 206 in cooperation with the display driver unit 204 superimposes a reference marker 220 (FIG. 7), constituting an alignment reference, for example a circular reference marker, on the display device 226 over the image 222 of the eye captured by the camera 112 and that is also displayed on the display device 116. In other examples, other shapes can be employed to form the reference marker, for example a number of individual points, a square shape, one or more arcs, possibly of different radii, or a horseshoe shape. In this example, the reference marker 220 is of fixed shape and size. However, in other examples the size of the reference marker 220 can, optionally, vary during use to provide an indication to the operator as to whether the apparatus 100 should be brought closer to or further away from thepatient's eye. The camera 112 also captures a reflection 224 by the eye of the light emitted by the light source 110.

Following either on-screen or previously learnt instructions, the operator makes small adjustments to the relative position between the pachymeter 100 and the patient's eye until the reflection 224 of the light source 110 is substantially within the reference marker 220 and an outer periphery of the reflection 224 is substantially as large as the reference marker 220. Additionally, the operator strives to ensure that the radius of the reflection 224 of the light source 110 is uniform. In this regard, by ensuring that the reflection 224 is substantially within the reference marker 220 and uniform, coarse X and Y alignment of the optical axis 202 of the pachymeter 100 relative to the patient's eye can be obtained. Thus, the reference marker 220 serves as a reference to facilitate manual alignment of the optical measurement system with the patient's eye. In this regard, other criteria can be employed with respect to the relative position of the reflection of the light source 110, for example the reference marker 220 can be sized such that it is a requirement to ensure that the reflection 224 of the light source 110 is outside the reference marker 220 and the operator strives to ensure that an inner periphery of the reflection 224 is substantially as large as the reference marker 220.

When the reflection 224 is not central with respect to the reference marker 220, the pachymeter 100 is misaligned with respect to the patient's eye in X and Y axis, and when the reflection 224 is not of a uniform radius, the pachymeter 100 is misaligned in pitch and yaw.

The confocal measurement unit 200 has a limited range of scanning. By ensuring that the reflection 224 is substantially the same size as the reference marker 220, for example they have a substantially common diameter, the distance of the pachymeter 100 to the anterior surface of the cornea of the patient's eye is sufficiently close for the scanning range of the confocal measurement unit 200 to extend through the cornea of the patient's eye.

Referring back to FIG. 6, once the controller 216 has determined, via the display driver unit 204, that the operator has selected to initiate measurement, the controller 216 monitors the alignment of the pachymeter 100 with respect to the patient's eye. In this regard, two different techniques to monitor (Step 402) X and Y alignment of the pachymeter 100, and in particular the confocal measurement unit 200, with the eye are described herein.

Turning to FIGS. 8 and 9, in a first example, the image capture unit 208 captures (Step 420) an image of the patient's eye and passes the image to the display driver unit 204 to be displayed by the display device 116 as described above. However, the image capture unit 208 also provides the image to the boundary analysis unit 210 which, under the control of the controller 216, analyses the image comprising the reflection 224 of the light source 110 in order to determine fidelity of shape and centrality of the reflection 224. Referring to FIG. 8, which is used here to assist in visualisation of the operations performed by the boundary analysis unit 210, the boundary analysis unit 210 firstly identifies (Step 422) a vertical midline 226 in the image provided by the image capture unit 208 and constituting a central reference line. The boundary analysis unit 210 analyses the image to identify pixels of high intensity, for example above a predetermined threshold value. Moving down the vertical midline 226, the boundary analysis unit 210 identifies (Step 424) a horizontal position below the region of high intensity 228, which is part of the reflection 224 of the light source 110 captured in the image of the eye. The horizontal position identified constitutes a horizontal starting position. The boundary analysis unit 210 then scans (Step 426) horizontally outwards from the midline 226 in a first direction, for example as illustrated as a first horizontal line portion 230, examining the illuminance of each pixel along the first horizontal line portion 230. For each pixel that is examined, the boundary analysis unit 210 determines (Step 428) whether the illuminance of the pixel exceeds a predetermined threshold value. If the illuminance of the pixel being examined does not exceed the predetermined threshold, an inner peripheral boundary 232 of the reflection of the light source 110 is deemed not to have been reached and the boundary analysis unit 210 continues to scan (Step 426) outwardly along the first horizontal line potion 230. Once the illuminance of a pixel along the first horizontal line portion 230 exceeds the predetermined illuminance threshold, the boundary analysis unit 210 considers the inner peripheral boundary 232 of the reflection 224 of the light source 110 to have been reached and the coordinates of the pixel are stored (Step 430). Thereafter, the boundary analysis unit 210 commences scanning (Step 432) from the midline 226 in an opposite direction to the first horizontal line portion 230, i.e. along a second horizontal line portion 234 in this example. The boundary analysis unit 210 examines the illuminance of each pixel along the second horizontal line portion 234. For each pixel that is examined, the boundary analysis unit 210 determines (Step 434) whether the illuminance of the pixel exceeds the predetermined threshold value. If the illuminance of the pixel being examined does not exceed the predetermined threshold, the inner peripheral boundary 232 of the reflection 224 of the light source 110 is deemed not to have been reached and the boundary analysis unit 210 continues to scan (Step 432) outwardly along the second horizontal line potion 234. In another example, the illuminance of pixels being analysed can be compared to the illuminance of respective neighbouring pixels in order to determine whether the illuminance of a given pixel being analysed has increased sufficiently to represent a boundary being reached. Once the illuminance of a pixel along the second horizontal line portion 234 exceeds the predetermined illuminance threshold, the boundary analysis unit 210 considers the inner peripheral boundary 232 of the reflection of the light source 110 to have been reached in the other direction and the coordinates of the pixel are stored (Step 436). Although, in this example, the vertical midline 226 has been employed as the central reference line, other lines can be employed, for example a horizontal midline or any other line, for example a diagonal line or a line subtending any other angle with respect to the horizontal or vertical, the scanning outwardly being in a direction perpendicular to the selected central reference line.

Thereafter, the boundary analysis unit 210 determines (Step 438) whether further measurements need to be made. If further measurements need to be made, the boundary analysis unit 210 increments (Step 440) the current horizontal position selected, for example to a second position 236, and the above-described steps (Steps 426 to 438) of identifying intersections of the horizontal line portions at the newly selected horizontal position 236 with the inner peripheral boundary 232 of the reflected light source 110 are repeated. Once the boundary analysis unit 210 determines that no further measurements need to be made, i.e. a sufficiently large sets of boundary points corresponding to the inner peripheral boundary 232 of the reflection 224 of the light source 110 have been obtained in order to enable modelling, for example by a suitable curve fitting technique, the boundary analysis unit 210 signals the curve fitting engine 212 to indicate that sufficient data has been acquired, and the curve fitting engine 212 commences to fit (Step 442) a circle, constituting a boundary line, to the data points stored by the boundary analysis unit 210 that corresponds to the inner peripheral boundary 232 of the reflected light source 110. In this regard, the distances to the boundary identified can be used to model the boundary line. The curve fitting engine 212 then tests (Step 444) the goodness of fit of the circle generated by the curve fitting engine 212 using any suitable goodness of fit test. If the curve fitting engine 212 determines (Step 446) that the fit is not adequate, the above process (Steps 420 to 444) is repeated until a curve is generated by the curve fitting engine 212 that is an adequate fit. When an adequate fit has been found, the boundary line can be assessed for centrality, fidelity of shape and optionally size. In this respect, the curve fitting engine 212 firstly provides the XY determination unit 214 with a centre coordinate and radius of the circle generated.

Turning to FIG. 10, the XY determination unit 214, upon receipt (Step 448) of the centre coordinate and radius of the circle generated, determines (Step 450) whether the circle is positioned sufficiently centrally with respect to the reference marker 220. If the circle is found not to be sufficiently centrally positioned, the controller 216 restarts the above-describes process (Steps 420 to 450) to fit a circle to the inner peripheral boundary 232 of the reflection 224 of the light source 110 as captured in a subsequent image by the camera 112. However, if the circle is found to be sufficiently centrally positioned, the XY determination unit 214 next determines (Step 452) the diameter of the circle generated. Thereafter, the XY determination unit 214 determines (Step 454) whether the diameter of the circle generated is within an acceptable numerical limit. In the event that the diameter of the circle generated is not of an acceptable size, the controller 216 restarts the above-describes process (Steps 420 to 454) to fit a circle to the inner peripheral boundary 232 of the reflection 224 of the light source 110 as captured in a subsequent image by the camera 112. However, if the diameter of the circle generated is found to be of an acceptable size, the controller 216 proceeds, in response to the assessment, to a confocal scanning stage (Step 404; FIG. 6), but also restarts the above-describes process (Steps 420 to 454) to fit a circle to the inner peripheral boundary 232 of the reflection 224 of the light source 110 as captured in a subsequent image by the camera 112.

In this regard, and referring back to FIG. 6, the controller 216 determines (Step 404) whether the confocal measurement unit 200 is already performing a confocal scan of the patient's eye. In the event that the confocal measurement unit 200 is not yet performing the confocal scan, the controller 216 instructs and confocal measurement unit 200 to initiate (Step 406) a confocal scan. However, if the controller 216 determines that the confocal measurement unit 200 is already performing the confocal scan, the controller 216 simply allows (Step 408) the confocal measurement unit 200 to continue performing the confocal scan.

Referring to FIG. 11, the confocal measurement unit 200 performs (Step 460) a confocal scan of the patient's eye along the confocal optical axis 202 thereof. The confocal scan can be in accordance with any suitable known technique, for example as described in UK patent number GB-B-2 508 368 and UK patent application number GB 2107470.3. In this regard, as mentioned above, the confocal measurement unit 200 is either already performing a confocal scan or one needs to be initiated. In either case, once the confocal scan has been performed, the output of the scan is provided to the confocal measurement processor unit 201. The confocal measurement process unit 201 then determines (Step 462) whether the output of the scan provided comprises any peaks. If the output of the scan does not comprise any peaks, the confocal measurement unit 200 repeats the confocal scan (Step 460) and this process is repeated (Steps 460 and 462) until the confocal measurement processor unit 201 determines that the output of the scan comprises at least one peak. When the confocal measurement processor unit 201 identifies at least one peak, the confocal measurement processor unit 201 processes the location of the peak within the output of the scan in order to determine (Step 464) one or more Z-positions of the confocal measurement unit 200 from the patient's eye. The confocal measurement processor unit 201 then determines (Step 466) whether the output of the scan comprises more than one peak. If the output of the scan only comprises one peak, the confocal measurement processor unit 201 provides (Step 468) the Z-position calculated to the display driver unit 204 for visual output by the display device 116 to the operator, and the confocal measurement processor unit 201 instructs the confocal measurement unit 200 to repeat the above-described confocal scanning process (Steps 460 to 466).

In the event that the output of the scan comprises more than one peak, the confocal measurement processor 201 determines (Step 470) whether the scan is valid, for example as described in UK patent number GB-B-2 451 443. Validation of the scan will be described later herein with reference to FIG. 12. If the scan is not found to be valid, the confocal measurement processor unit 201 provides (Step 468) the Z-positions calculated to the display driver unit 204 for visual output by the display device 116 to the operator, and the confocal measurement processor unit 201 instructs the confocal measurement unit 200 to repeat the above-described confocal scanning process (Steps 460 to 470). However, if the scan is found to be valid, the output of the scan is stored (Step 472) and the controller 216 determines (Step 474) whether a sufficient number of valid confocal scans have been acquired. If the number of valid scans acquired is insufficient, the confocal measurement processor unit 201 provides (Step 468) the calculated Z-positions to the display driver unit 204 for visual output by the display device 116 to the operator, and the confocal measurement processor unit 201 instructs the confocal measurement unit 200 to repeat the above-described confocal scanning process (Steps 460 to 474).

If a sufficient number of confocal scans has been acquired, the controller 216 instructs the confocal measurement processor unit 201 to calculate (Step 410; FIG. 6) a thickness of the cornea of the patient's eye using any suitable technique, for example an averaging technique. This value can then be displayed by the display device 116 or averaged with multiple corneal thickness calculations before being displayed by the display device 116.

Referring to FIG. 12, in order to validate the scan generated by the confocal measurement unit 200, the confocal measurement processor unit 201 firstly awaits (Step 480) the completion of a confocal scan. Once the confocal measurement processor unit 201 has conformed that the confocal scan has been completed, the confocal measurement processor unit 201 then accesses (Step 482) X and Y alignment data previously generated by the X and Y determination unit 214 and obtained from the controller 216 corresponding to a time when the confocal scan was performed. The confocal measurement processor unit 201 then checks (Step 484) whether the confocal scan performed by the confocal measurement unit 200 is valid, for example checking that the amplitudes of the peaks are within respective expected ranges, checking the Full Width at Half Maximum (FWMH) values of the peaks are within respective expected ranges, and the distance between the peaks corresponds to a thickness that is within an expected range of thicknesses. In the event that the confocal scan is not found to be valid, the verification of the confocal scan is halted and the processing of the confocal scan returns to the calculation and provision (Step 468; FIG. 11) of a Z-position to the display driver unit 204 described above. Otherwise, the confocal measurement processor unit 201 proceeds to determine (Step 486) whether the confocal measurement unit 200 was effectively aligned in X and Y axes at the time of scanning. If the alignment is found not to be acceptable, the verification of the confocal scan is halted and the processing of the confocal scan returns to the calculation and provision (Step 468; FIG. 11) of a Z-position to the display driver unit 204 described above. Otherwise, the confocal measurement processor unit 201 tags (Step 488) the confocal scan as valid. Thereafter, the confocal measurement processor unit 201 determines (Step 490) whether validation of further scans is required. If further validation is required, the confocal measurement processor unit 201 repeats the above-described process (Step 480 to 490). Otherwise, the indication that the focal scan is valid is used by the confocal measurement processor unit 201 when the confocal measurement processor unit 201 stores (Step 472; FIG. 11) the confocal scan prior to determining (Step 474; FIG. 11) whether a sufficient number of valid confocal scans have been collected to enable a central confocal thickness to be calculated (Step 410; FIG. 6). As such, it can be seen that the optical measurement system makes a plurality of distance measurements, for example to the reflective target, such as the patient's eye. Also, it can be seen that a plurality of alignment assessments is made respectively corresponding to the plurality of measurements made, which in this example are distance measurements, and measurements made when the optical measurement system is adequately aligned are selected. In this regard, each alignment assessment of the plurality of alignment assessments corresponds to an assessment of a state of alignment of the optical measurement system with respect to the reflective target.

In another example (FIGS. 13 to 16), an alternative technique to the technique of FIGS. 8 and 9 to determine X and Y alignment of the confocal measurement unit 200 can be employed. In such an alternative technique, the light source 100 can be any suitable shape capable of providing an optimally distinctive shape not prone to confusion with other shapes, for example instead of being annular, the light source 100 can possess the substantially horseshoe-like shape mentioned above. In this regard and referring back to FIG. 6, while the confocal scanning unit 200 is scanning (Steps 406/408), the alignment of the confocal scanning unit 200 with the patient's eye is performed as follows.

The image capture unit 208 captures (Step 500) an image of the patient's eye and passes the image to the display driver 204 for display by the display device 116 as described above. However, the image capture unit 208 also provides the image to the boundary analysis unit 210 which, under the control of the controller 216, analyses the image comprising the reflection 224 of the light source 110. In this example, the curve fitting engine 212 and the X and Y determination unit 214 are not employed.

Referring to FIG. 14, this technique employs a first pair of sets of pixel positions 600, a second pair of sets of pixel positions 602 and a third pair of sets of pixel positions 604, constituting a plurality of sets of substantially parallel pixel positions. The first pair of sets of pixel positions 600 comprises a first set of vertically arranged pixel positions 606 and a second set of vertically arranged pixel positions 608, the first and second sets of vertically arranged pixel positions 606, 608 being, in this example, substantially parallel with respect to each other. In this example, the first pair of sets of pixel positions 600 are disposed in an outermost position relative to the second and third pairs of sets of pixel positions 602, 604. The second pair of sets of pixel positions 602 comprises a third set of vertically arranged pixel positions 610 and a fourth set of vertically arranged pixel positions 612, the third and fourth sets of vertically arranged pixel positions 610, 612 being, in this example, substantially parallel with respect to each other. The third pair of sets of pixel positions 604 comprises a fifth set of vertically arranged pixel positions 614 and a sixth set of vertically arranged pixel positions 616, the fifth and sixth sets of vertically arranged pixel positions 614, 616 being, in this example, substantially parallel with respect to each other. In this example, the third pair of sets of pixel positions 604 is disposed at an innermost position relative to the first and second pairs of sets of pixel positions 600, 602. The plurality of sets of substantially parallel pixel positions therefore has respective predetermined spacings therebetween. In this example, the second pair of sets of pixel positions 602 is disposed between the first and third pairs of sets of pixel positions 600, 604. Additionally, in this example, the first, second and third pairs of sets of pixel positions 600, 602, 604 are vertically offset with respect to each other. For example, the first pair of sets of pixel positions 600 is vertically offset with respect to the second pair of sets of pixel positions 602, and the third pair of sets of pixel positions 604 is offset with respect to the second of sets of pixel positions 602. The objective, when assessing X and Y alignment of the confocal measurement unit 200, is for the reflection 224 of the light source 110 present in the image captured by the image capture unit 208 using the camera 112 to be disposed between the first and third pairs of sets of pixel positions 600, 604 in the captured image. The second pair of sets of pixel positions 602 should be substantially in the middle of the area bounded by the inner and outer diameters of the ring-like shape of the reflection 224 of the light source 110. As can be seen, the plurality of sets of substantially parallel pixel positions 600, 602, 604 are arranged to correspond to expected locations of a first peripheral side 618 of the reflection 224 of the light source 110, a second peripheral side 620 of the reflection 224 of the light source 110, and a position 622 between the first and second peripheral sides 618, 620. The second peripheral side 620 is disposed opposite the first peripheral side 618. In this regard, the expected locations of the first peripheral side 618, the second peripheral side 620 and the position 622 in-between the first and second peripheral sides 618, 620 correspond to an aligned state of the optical measurement system with the reflective target. In this example, each set of pixel positions of the first, second and third pairs of sets of pixel positions 600, 602, 604 are, in addition to extending vertically, disposed in spaced relation. Although in this example, each set of pixel positions of the first, second and third pairs of sets of pixel positions 600, 602, 604 extend vertically, other orientations are contemplated, for example horizontally. In this example, the first pair of sets of pixel positions 600 constitutes an outer boundary set of pixel positions, the second pair of sets of pixel positions 602 constitutes an inner boundary set of pixel positions, and the third pair of sets of pixel positions 604 constitutes an intermediate set of pixel positions disposed between the first and second sets of pixel positions.

For each pair of sets of substantially parallel pixel positions making up the plurality of sets of substantially parallel pixel positions, a given set of substantially parallel pixel positions comprises a first subset of pixel positions and a second subset of pixel positions, for example in the case of the first pair of sets of pixel positions 600, the first and second sets of vertically arranged pixel positions 606, 608 constitute the first and second subsets of pixel positions that are substantially parallel with respect to each other. In this example, the first and second subsets of pixel positions, for example the first and second, the third and fourth, and the fifth and sixth sets of vertically arranged pixel positions, 606, 608, 610, 612, 614, 616, are respectively arranged linearly.

Returning back to FIG. 13, the boundary analysis unit 210 firstly attempts to determine (Step 502) whether the luminosity at a sufficient number of pixel positions of the first pair of sets of pixel positions 600, which are the outermost sets of pixel positions, is less than a predetermined threshold value, i.e. the position and size of the reflection 224 of the light source 110 is not such that the pixels of the reflection 224 of the light source 110 overlap the first pair of sets of pixel positions 600. This is just one example of a predetermined illuminance threshold criterion and any other suitable criterion can be employed in other examples. The test is a simple pass/fail test and so in the event that the test is failed, the boundary analysis unit 210 instructs the image capture unit 208 to capture (Step 500) another image and the determination step in relation to the first pair of sets of pixel positions (Step 502) is repeated. However, if the reflection 224 of the light source 110 is between the first pair of sets of pixel positions 600, then the size of the reflection 224 of the light source 110 is not too large.

The test (Step 502) comprises the following assessment for each pixel position of the first pair of sets of pixel positions 600. Referring to FIG. 15, the boundary analysis unit 210 selects (Step 600) a first pixel position from the first pair of sets of picks up positions 600. The boundary analysis unit 210 then determines (Step 602) whether the illuminance at the selected pixel position is less than the predetermined threshold value mentioned above. In the event that the illuminance at the selected pixel position is less than the predetermined threshold value, the pixel position is tagged (Step 604) for being compliant with this illuminance test. The boundary analysis unit 210 then determines (Step 606) whether further pixel positions in the first pair of sets of pixel positions 600 remain to be tested. Likewise, if the illuminance of the selected pixel position is not less than the predetermined threshold value, then the boundary analysis unit 210 also proceeds to determine (Step 606) whether further pixel positions in the first pair of sets of pixel positions 600 remain to be tested. If further pixel positions in the first pair of sets of pixel positions 600 remain to be tested, a variable keeping track of the current pixel position being tested is incremented (Step 608) so that a subsequent pixel position in the first pair of sets of pixel positions 600 can be tested next. The process is repeated (Steps 600 to 608) until all pixel positions in the first pair of sets of pixel positions 600 have been tested. Thereafter, the boundary analysis unit 210 determines (Step 610) whether the number of pixel positions, tagged as having an illuminance less than the predetermined threshold value, is greater than a predetermined number. In the event that the threshold number has been exceeded, the test is deemed to have been passed, whereas if the number of tagged pixel positions is less than the predetermined number, the test is deemed to have been failed, and the boundary analysis unit 210 instructs the image capture unit 208 to capture (Step 500; FIG. 13) another image and the determination step in relation to the first pair of sets of pixel positions (Step 502) is repeated.

Referring back to FIG. 13, in the event that the illuminance at a sufficient number of the pixel positions of the first pair of sets of pixel positions 600 is less than the predetermined threshold value, i.e. the above-described test has been passed, the boundary analysis unit 210 proceeds to a further test in relation to the third pair of sets of pixel positions 604. In this respect, the boundary analysis unit 210 next determines (Step 504) whether the illuminance at a sufficient number of pixel positions of the third pair of sets of pixel positions 604 is less than the predetermined threshold value mentioned above. In this regard, and referring back to FIG. 15, the test performed in relation to the first pair of sets of pixel positions 600 is now applied to the third pair of sets of pixel position 604. As such, the boundary analysis unit 210 selects (Step 600) a first pixel position from the third pair of sets of pixel positions 604. The boundary analysis unit 210 then determines (Step 602) whether the illuminance at the selected pixel position is less than the predetermined threshold value mentioned above. In the event that the illuminance at the selected pixel position is less than the predetermined threshold value, the pixel position is tagged (Step 604) for being compliant with this illuminance test. The boundary analysis unit 210 then determines (Step 606) whether further pixel positions in the third pair of sets of pixel positions 604 remain to be tested. Likewise, if the illuminance of the selected pixel position is not less than the predetermined threshold value, then the boundary analysis unit 210 also proceeds to determine (Step 606) whether further pixel positions in the third pair of sets of pixel positions 604 remain to be tested. If further pixel positions in the third pair of sets of pixel positions 604 remain to be tested, the variable keeping track of the current pixel position being tested is incremented (Step 608) so that a subsequent pixel position in the third pair of sets of pixel positions 604 can be tested next. The process is repeated (Steps 600 to 608) until all pixel positions in the third pair of sets of pixel positions 604 have been tested. Thereafter, the boundary analysis unit 210 determines (Step 610) whether the number of pixel positions, tagged as having an illuminance less than the predetermined threshold value, is greater than a predetermined number. In the event that the threshold number has been exceeded, the test is deemed to have been passed, whereas if the number of tagged pixel positions is less than the predetermined number, the test is deemed to have been failed, and the boundary analysis unit 210 instructs the image capture unit 208 to capture (Step 500) another image and the determination step in relation to the first and third pairs of sets of pixel positions (Step 502 and 504) are repeated.

Referring back to FIG. 13, in the event that the illuminance at a sufficient number of pixel positions of the third pair of sets of pixel positions 604 is less than the predetermined threshold value, i.e. the above-described test has been passed, the boundary analysis unit 210 proceeds to test (Step 506) whether the illuminance at the pixel positions of the second pair of sets of pixel positions 602 is greater than another predetermined threshold value. Referring to FIG. 16, the boundary analysis unit 210 selects (Step 620) a first pixel position from the second pair of sets of pixel positions 602. The boundary analysis unit 210 then determines (Step 622) whether the illuminance at the selected pixel position is greater than the predetermined threshold value mentioned above. In the event that the illuminance at the selected pixel position is greater than the predetermined threshold value, the pixel position is tagged (Step 624) for being compliant with this illuminance test. The boundary analysis unit 210 then determines (Step 626) whether further pixel positions in the second pair of sets of pixel positions 602 remain to be tested. Likewise, if the illuminance of the selected pixel position is not greater than the predetermined threshold value, then the boundary analysis unit 210 also proceeds to determine (Step 626) whether further pixel positions in the second pair of sets of pixel positions 602 remain to be tested. If further pixel positions in the second pair of sets of pixel positions 602 remain to be tested, a variable keeping track of the current pixel position being tested is incremented (Step 628) so that a subsequent pixel position in the second pair of sets of pixel positions 602 can be tested next. The process is repeated (Steps 620 to 628) until all pixel positions in the second pair of sets of pixel positions 602 have been tested. Thereafter, the boundary analysis unit 210 determines (Step 630) whether the number of pixel positions, tagged as having an illuminance greater than the predetermined threshold value, is greater than a predetermined number. In the event that the threshold number has been exceeded, the test is deemed to have been passed, whereas if the number of tagged pixel positions is less than the predetermined number, the test is deemed to have been failed. Referring back to FIG. 13, in the event that the test of the second pair of sets of pixels 602 results in a fail, the boundary analysis unit 210 instructs the image capture unit 208 to capture another image (Step 500) another image and the determination steps in relation to the first, second and third pairs of sets of pixel positions (Step 502 to 506) are repeated. However, if the test of the second pair of sets of pixels 602 results in a pass, the boundary analysis unit 210 reports the result of the tests back to the controller 216 indicating that X and Y alignment has been adequately achieved and the controller 216 returns to determining (Step 404; FIG. 6) whether confocal scanning is being performed as described above in relation to the first example. It should be appreciated that the thresholds described above can be set or adjusted by reference to a number of pixels of the image captured, for example an average of the illuminances of the number of pixels. In some examples, all illuminances of all the pixels in the image can be taken into account. This is sometimes useful where the image is brighter owing to ambient light levels or where the parts of the eye appear brighter or darker than other eyes for which the apparatus is calibrated. In such circumstances, and others, the thresholds can be adjusted so that performance of the apparatus 100 is not degraded.

The skilled person should appreciate that the above-described implementations are merely examples of the various implementations that are conceivable within the scope of the appended claims. In this regard, although the above-described alignment and measurement system employs a confocal measurement technique to implement axial measurement for the determination of the distance from the measurement system to a measurement target, the skilled person should appreciate that any suitable axial measurement technique can be employed, for example an interferometric axial measurement technique.

What is claimed is:

1. A handheld optical measurement apparatus, the apparatus comprising:
an optical measurement system comprising an optical reception path;
a display device;
a housing comprising a longitudinal measurement axis and the optical measurement system, the optical reception path being coaxial, at least in part, with the longitudinal measurement axis; and
an alignment system comprising:
an extended light source configured as a predetermined shape and mounted on the housing and disposed off-axis with respect to the longitudinal measurement axis, the extended light source being configured to illuminate, when in use, a reflective target;
an optical sensor device mounted on the housing off-axis relative to the longitudinal measurement axis and configured to receive, when in use, light reflected by the reflective target and to capture an image comprising the light; and
a processing resource operably coupled to the optical sensor device and the display device; wherein
the processing resource is configured to display on the display device the image captured by the optical sensor device and substantially contemporaneously display an alignment reference on the display device, the alignment reference being fixed in position, and providing, when in use, a reference to facilitate manual alignment of the optical measurement system with the reflective target; and
the processing resource is configured to assess alignment of the optical measurement system with respect to fidelity of shape and centrality of the reflected light in the image captured by the optical sensor device.

2. The apparatus according to claim 1, wherein the optical measurement system is configured to make a measurement in response to the assessment of alignment.

3. The apparatus according to claim 1, wherein the processing resource is arranged to analyse the image comprising the reflected light captured by the optical sensor device and to identify a plurality of boundary pixels of the reflected light.

4. The apparatus according to claim 3, wherein the processing resource is configured to define a central reference line within the image comprising the reflected structured light captured and to measure a plurality of perpendicular distances from the central reference line to the plurality of boundary pixels, respectively.

5. The apparatus according to claim 3, wherein the processing resource is arranged to model a boundary line defined by the plurality of boundary pixels and assess the centrality, size and fidelity of shape of the boundary line.

6. The apparatus according to claim 1, wherein the processing resource is configured to analyse a plurality of sets of substantially parallel pixel positions, the plurality of sets of parallel pixel positions having respective predetermined spacings therebetween.

7. The apparatus according to claim 6, wherein the plurality of sets of substantially parallel pixel positions are offset with respect to each other.

8. The apparatus according to claim 6, wherein the plurality of sets of substantially parallel pixel positions are arranged to correspond to expected locations of a first peripheral side of the reflected light, a second peripheral side of the reflected structured light and a position between the first and second peripheral sides, the second peripheral side being opposite the first peripheral side.

9. The apparatus according to claim 6, wherein the plurality of sets of substantially parallel pixel positions comprises:
an outer boundary set of pixel positions;
an inner boundary set of pixel positions; and
an intermediate set of pixel positions between the first and second boundary sets of pixel positions.

10. The apparatus according to claim 6, wherein the processing resource is configured to analyse illuminance of pixel positions of each set of the plurality of sets of substantially parallel pixel positions in order to determine whether the pixels of the each set of the plurality of sets of substantially parallel pixel positions satisfy a respective predetermined illuminance threshold criterion.

11. The apparatus according to claim 1, wherein the optical measurement system is configured to make a plurality of distance measurements.

12. The apparatus according to claim 11, wherein the plurality of measurements is a plurality of distance measurements to the reflective target.

13. The apparatus according to claim 12, further comprising:
the optical sensor device configured to capture a plurality of images comprising the reflected light;
the processing resource configured to make a plurality of respective assessments of alignment of the optical measurement system with respect to fidelity of shape and centrality of the reflected light of the plurality of images captured, the plurality of assessments of alignment respectively corresponding to the plurality of distance measurements; wherein
the processing resource is configured to select a measurement of the plurality of measurements in response to an alignment assessment of the plurality of alignment assessments, the alignment assessment corresponding to the measurement of the plurality of measurements.

14. The apparatus according to claim 11, further comprising:
the processing resource configured to indicate the plurality of distance measurements.

15. A method of aligning a handheld optical measurement apparatus with a reflective target, the method comprising:
an extended light source of a predetermined shape illuminating the reflective target from an off-axis position with respect to a longitudinal measurement axis of a housing of the handheld optical measurement apparatus;
receiving light reflected from the reflective target;
capturing an image comprising the reflected light received using an optical sensor device mounted on a housing of the handheld optical measurement apparatus and off-axis relative to the longitudinal measurement axis of the housing;
displaying the image captured by the optical sensor device and substantially contemporaneously displaying an alignment reference with the image, the alignment reference being fixed in position, and providing, when in use, a reference to facilitate manual alignment of an optical measurement system of the handheld optical measurement apparatus with the reflective target; and
assessing alignment of the optical measurement system with respect to fidelity of shape and centrality of the reflected light in the image captured by the optical sensor device.
